(19) United States
(12) Patent Application Publication
(10) Pub. No.: US 2006/0020156 A1
(43) Pub. Date: Jan. 26, 2006

(54) Therapeutic device

(76) Inventor: Vishnu Shanker Shukla, Jeddah (SA)

Correspondence Address: NIXON & VANDERHYE, PC, 901 NORTH GLEBE ROAD, 11TH FLOOR, ARLINGTON, VA, 22203, US

(21) Appl. No.: 11/169,330

(22) Filed: Jun. 29, 2005

(30) Foreign Application Priority Data
Jul. 5, 2004 (GB) 0415112.2

(51) Int. Cl. A61N 5/00

(52) U.S. Cl. 600/3

(57) ABSTRACT

The present invention relates generally to methods and devices for the treatment of cancers in viscous organs such as the bladder by brachytherapy, such as high dose rate brachytherapy. The invention provides a brachytherapy device consisting of a double balloon system in which a smaller dosing balloon is present within a larger multi-purpose balloon, and more specifically an apparatus comprising: (a) a flexible catheter for providing an access passageway into the body, which catheter has a distal end and a proximal end, (b) one or more brachytherapy tubes carried within the catheter for receiving the radioactive source at the proximal end of the catheter and transferring the source into the cavity, (c) an outer inflatable balloon element mounted at the distal end of the catheter for securing the catheter in the cavity and engaging the internal surface of the cavity and optionally distending the inner surface of the cavity, (d) an inner inflatable balloon element mounted within the outer balloon element for positioning the brachytherapy tubes within the outer balloon element, (e) a first fluid tube carried within the catheter for inflating the outer balloon element, (f) a second fluid tube carried within the catheter for inflating the inner balloon element.

### TECHNICAL FIELD

The present invention relates generally to methods and devices for the treatment of cancers in viscous organs such as the bladder (e.g. bladder cancer, urinary bladder cancer) by brachytherapy.

### BACKGROUND ART

Surgery is a common treatment for bladder cancer, with various types being used including transurethral resection, radical cystectomy, segmental cystectomy and urinary diversion. Radiation therapy has also been used to treat bladder cancer. External radiation has been applied using a large machine outside the body to aim radiation at the tumor area, generally for 5 days a week for 5 to 7 weeks as an inpatient or outpatient. This may have the side effect of severe fibrosis, making subsequent operation very difficult and multiplying postoperative complications.

For internal radiation, the doctor places a small container of a radioactive substance into the bladder through the urethra or through an incision in the abdomen. This is a surgical procedure and the patient stays in the hospital for several days during this treatment.

Publications describing such techniques include Friedman M, Lewis L. G. “Irradiation of Ca of bladder by a central intracavitary radium or cobalt 60 source” American Journal of Roentgenology and Rad Therapy Nuclear medicine, 79:6-31 1958; Clarence B. Hewitt, Jan F. Babiszewski and Antonio R. Antunez, “Update on intracavitary radiation in the treatment of bladder tumours” 1981, The Journal of Urology, Vol 126, Part 3 pages 323 325 and “Combined intracavitary and external beam irradiation for superficial transitional cell carcinoma of the bladder: an alternative to cystectomy for patients with recurrence after intravesical chemotherapy” Kenneth J. Russell, Wui-Jin Koh, Anthony H. Russel, Brian R. Griffin, Katherine L. Markette, Daphne Y, Tong and Thomas W. Griffin, Univ of Washington, Journal of Urology, 141:30-32 1989.

WO 03/051450 and U.S. 2003/0032851 describe balloon catheters.

### DISCLOSURE OF THE INVENTION

Briefly, the invention provides a brachytherapy device consisting of a double balloon system in which a smaller dosing balloon is present within a larger multi-purpose balloon. This can be used in viscous organs such as the bladder in an outpatient procedure without cutting of tissue or anaesthetic. The device can be prepared, in the light of the disclosure herein, by modification of existing HDR brachytherapy applicator devices and bladder irrigation catheters. The brachytherapy applicator or bladder irrigation catheter provides a means for supporting smaller catheters for inflation of each balloon and passage of radiation sources. It also allows smooth passage of these smaller catheters into and out of the viscous organ.

“Brachytherapy” is a technique which involves placing a source of radiation in proximity to a tumor and employs radioactive plaques, needles, tubes, wires, or small “seeds” made of radionuclides. These radioactive materials are placed over the surface of the tumor or implanted within the tumor, or placed within a body cavity surrounded by the tumor. As a result the radiation doses to the outside of the organ being treated are very low in comparison to external beam radiotherapy, with correspondingly reduced side effects on the surrounding normal tissue, making the tissues more amenable to a later operation, should that be required.

In one particular variant of brachytherapy, termed “HDR” (high dose rate) brachytherapy, thin catheters are first placed in the tumor. The catheters are then connected to an HDR loader. This machine contains a single highly radioactive iridium pellet at the end of a wire. The pellet is pushed into each of the catheters one by one under computer control. The computer controls how long the pellet stays in each catheter, and where along the catheter it should pause to release its radiation. With a few well placed catheters in the tumor, HDR brachytherapy can provide a very precise treatment that takes only a few minutes. After a series of treatments, the catheters are removed, and there are no radioactive seeds left in the body.

Applicator devices for performing HDR brachytherapy in certain tissues are known. For example U.S. publication 2003/0153803 describes a modified Fletcher-Suit tandem tube applicator which includes a balloon which can be inflated to both positionally secure the applicator within the vaginal canal and to distend the confronting vaginal wall thereby increasing the distance of such tissue from the radioactive source contained in the tandem tube of applicator and correspondingly reducing radiation damage to nearby tissues and organs such as the rectum and bladder. U.S. Pat. No. 5,562,594 describes an applicator system for brachytherapy of the uterine cervix.

HDR brachytherapy has previously been used for the treatment of cancers e.g. of the prostate, breast, lung, oesophagus, cervix, uterus, anus, bile duct, head, neck and tongue. However it has only been used sparingly in the bladder because of the complicated surgical procedures involved.

Thus in one aspect the invention provides an apparatus for applying therapeutic radiation emitted from a radiation source to a predetermined region of tissue within a viscous organ cavity in the body (for example, urinary bladder) of a living being, said apparatus comprising:
(a) a flexible catheter for providing an access passageway into the body, which catheter has a distal end and a proximal end,
(b) one or more brachytherapy tubes carried within the catheter for receiving the radioactive source at the proximal end of the catheter and transferring the source into the cavity,
(c) an outer inflatable balloon element mounted at the distal end of the catheter for securing the catheter in the cavity and engaging the internal surface of the cavity and optionally distending the inner surface of the cavity,
(d) an inner inflatable balloon element mounted within the outer balloon element for positioning the brachytherapy tubes within the outer balloon element,
(e) a first fluid tube carried within the catheter for inflating the outer balloon element,
(f) a second fluid tube carried within the catheter for inflating the inner balloon element.

The first and second fluid tubes may also be used for deflation of their respective balloons. Alternatively, the inner and outer balloons may be joined to one or more other fluid tubes for deflation.

The invention further provides a (brachytherapy) method of applying therapeutic radiation within a viscous organ cavity within the body of a living being, said method comprising:
(i) providing an apparatus as described above,
(ii) inserting the distal end of the catheter into a viscous organ cavity,
(iii) inflating the outer balloon such that its outer surface contacts the internal surface of the organ cavity,
(iv) partially inflating the inner balloon such as to distribute the or each brachytherapy tube within the outer balloon,
(v) transferring a radioactive source to a pre-determined position within the or each brachytherapy tube in the organ cavity for a pre-determined period of time such as to provide a targeted dosage of radiation to all or part of the internal surface of the organ cavity and/or the wall thickness of this organ cavity.

The balloons may then be deflated and the catheter removed.

The invention further provides the apparatus or kit (below) for use in such methods of treatment.

Calculation and insertion of the appropriate targeted dosage, and inflation (and deflation) of the balloons with appropriate fluids can be provided in conventional manner and systems for doing this do not per se form part of the present invention. The calculation may include providing imaging information about the volume of the organ cavity and the position of the brachytherapy tubes within it, and applying appropriate algorithms based, for example, on the nature of the radioactive source, the size and nature of the target cancerous tissue, the attenuation of the fluid in the outer balloon and so on. Thus a typical system may include an imaging apparatus, a microprocessor and control panel, and an afterloader for positioning the radioactive source. Such systems include, for instance, the Varisource 200/200t (from Varian Medical Systems UK Ltd., Brachytherapy, Crawley, West Sussex, UK) and the MCP afterloading system (from MCP Medical International GmbH, Kaninchenborn 24-28. D-23560, Luebeck, Germamy). The balloons may be inflated/deflated using any type of syringe, provided that the dispensing end of the syringe can engage with the respective fluid tubes e.g. a syringe with a “Luer” lock sized distal connector.

Some particular features of the invention will now be discussed in more detail:

For definitional purposes and as applicable, the tube end which enters the body is referred to as “distal” and the end accessible for manipulation by the healthcare provider is referred to as “proximal” (typically projecting external of the patient's body).

By “viscous” organ is meant any of the main internal organs of the body or a tumour bed within one of these organs. The invention is particularly applicable to those in the abdomen e.g. bladder, lungs, rectum, stomach.

The flexible catheter will be of any conventional tissue-compatible material e.g. soft latex or PVC, and preferably has an external diameter of about 8 mm (e.g. a diameter suitable to enter the urethra or any other organ), for example 7.7 mm (23 French (Charriere scale))

The brachytherapy tubes will generally be formed of fine catheters having an internal diameter suitable to receive radioactive sources of known type, e.g., a diameter suitable to traverse the radioactive point source, e.g. 0.6-1.0 mm. Such sources may be provided in the form of cylinders, cables or wires incorporating pellets of e.g. Cs-137 or Ir-192. The tubes may be present in the catheter and/or balloon lumen or wall. Any number may be used, provided they can be spaced around the internal balloon element e.g. 3, 4, 5, 8, 10 or more. Typically the tubes will terminate at their proximal end in attachment-means suitable for engaging a radioactive source loader e.g. a “Luer” lock.

Outer balloon elements meeting the requirements of the invention may be composed of any suitable medical grade elastomer that provides physical strength and elasticity characteristics required to practice the invention. They are mounted on the apparatus for the purposes of insertion into the cavity, and in fluid communication with the respective fluid tubes.

The outer balloon will typically be inflatable to at least a volume sufficient to contact and enlarge or distend the organ in question e.g. typically 300, 400, or 500 ml or more for use in the bladder. This is particularly important in the bladder since the “floppy” nature of the empty bladder makes targeting of radiation difficult.

The inner balloon will typically be inflatable to at least a volume sufficient to separate and distribute the distal ends of the brachyotherapy tubes e.g. between 2-10 ml. The inflation should provide a smooth ovoid profile to avoid kinking the brachyotherapy tubes.

The fluid for inflating the outer balloon is preferably non-radiation attenuating and may be chosen for compatibility with imaging systems—it may for example be air, water or diluted radio-opaque fluid.

If the fluid used to inflate the outer balloon does not provide sufficient contrast to allow it to be distinguished from the surrounding tissue by the imaging system (i.e. the fluid is ‘tissue equivalent’), preferably the outer balloon is made from a radio-opaque material.

Typically the first and second fluid tubes will terminate at their proximal end in attachment-means suitable for engaging conventional fluid sources or reservoirs to communicate the fluids, under pressure, therefrom e.g. “foley” catheter valves.

The fluid source or reservoir preferably should comprise means for the fluid source or reservoir to engage with the first and/or second fluid tubes e.g a “Luer” lock sized distal connector.

The balloons preferably affixed directly to the distal ends of the fluid tubes e.g. with an appropriate bonding adhesive such as Loctite or other cyanoacrylates in a manner to provide adequate adhesion and to minimize exposure of the adhesive in situ.

The catheter and/or balloons may be disposable and/or sterilisable.

Thus the invention also provides a kit for applying therapeutic radiation emitted from a radiation source to a predetermined region of tissue within a viscous organ cavity in the body of a living being, said kit comprising:
(a) a flexible catheter for providing an access passageway into the body, which catheter has a distal end and a proximal end,
(b) one or more brachytherapy tubes carried within the catheter for receiving the radioactive source at the proximal end of the catheter and transferring the source into the cavity,
(c) a first fluid tube carried within the catheter for inflating an outer balloon element,
(d) a second fluid tube carried within the catheter for inflating an inner balloon element, plus one or both of:
(e) an outer inflatable balloon element for mounting at the distal end of the catheter for securing the catheter in the cavity and engaging the internal surface of the cavity,
(f) an inner inflatable balloon element mounted within the outer balloon element for positioning the brachytherapy tubes within the outer balloon element.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1: Shows an embodiment of present invention with the balloon in inflated state (longitudinal section), showing outer balloon (11), inner balloon with catheter (10), passage for flow of urine (16), relative point source radioactive brachytherapy source to be passed in tubes (12, 13, 14, 15), outer opening of tube (16) to be attached to urine drainage bag, Luer lock fitting one way valve (17, 18) for inflating the outer balloon (11) and inner balloon (10), and Luer lock ends (19, 21).

FIG. 2: shows a longitudinal section view of an embodiment of the present invention with the outer balloon (11) and inner balloon (10) in the inflated state, urine flow opening (16), radioactive source passage tubes (12, 13, 14, 15) terminating separately at the distal ends of inner balloon (10), with connecting tubes (17, 18) to inflate the outer and inner balloons (11, 10).

FIG. 3: shows a lower proximal end view of the embodiment of the present invention which is the part of the same embodiment shown in FIG. 2, showing outer wall (8) of the complete embodiment; catheters (12, 13, 14, 15) for movement of radioactive relative point source into the inner balloon (10); lower end connection (20) to urine bag communicating with urine flow tube (16); the Luer lock one way valve (17) connected to the tube & outer balloon (11); valve (18) connected to the inner balloon (10); Luer lock fitting lower ends (19, 21).

FIG. 4: Shows a transverse section view of the embodiment of the present invention through the middle of the inner and outer balloon (8, 10), urine drainage line (16), radioactive transfer tubes (12, 13, 14, 15) in the wall of the inner balloon (10) wall.

FIG. 5: Shows a transverse section view of the embodiment of the present invention with outer wall (8), urine drainage tube (16), outer balloon inflating tube connection (17), inner balloon inflating tube connection (18), and radioactive source transfer tubes (12, 13, 14, 15).

FIG. 6: Shows a flow chart illustrating one embodiment of the catheter in use.

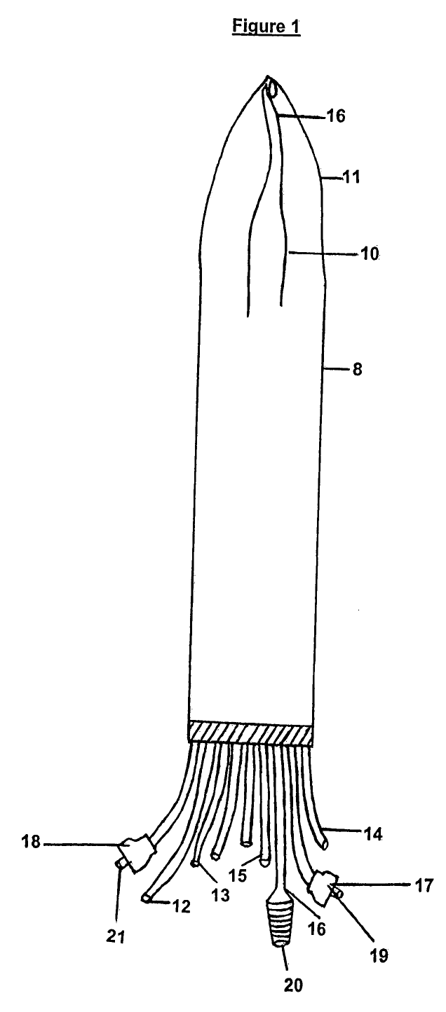

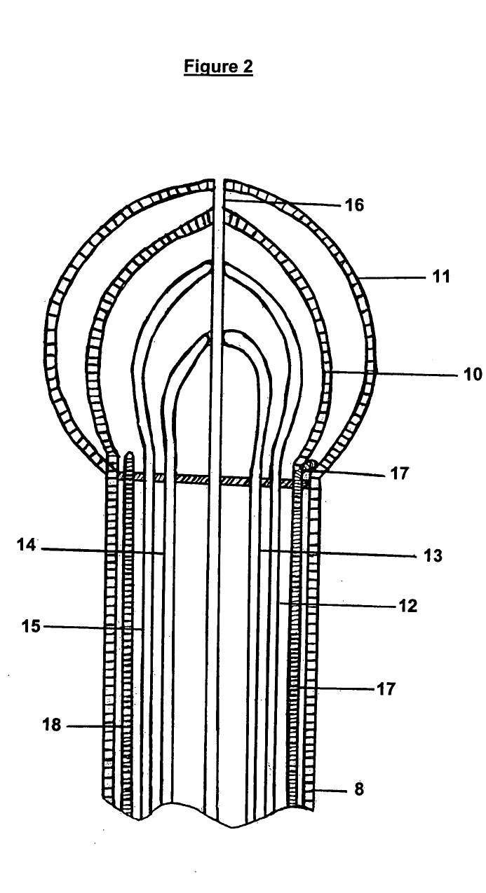

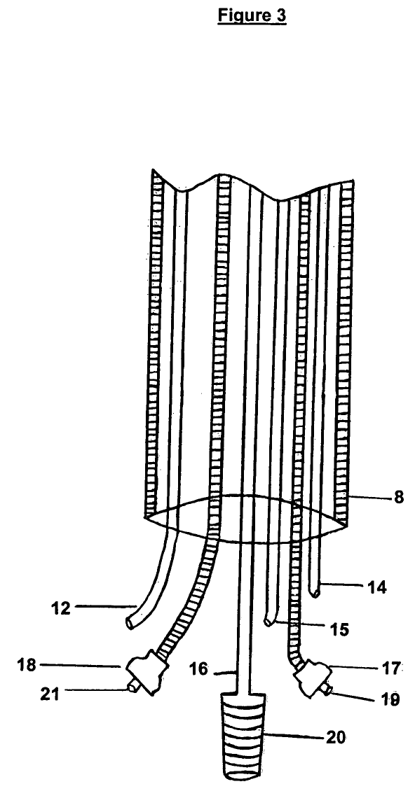

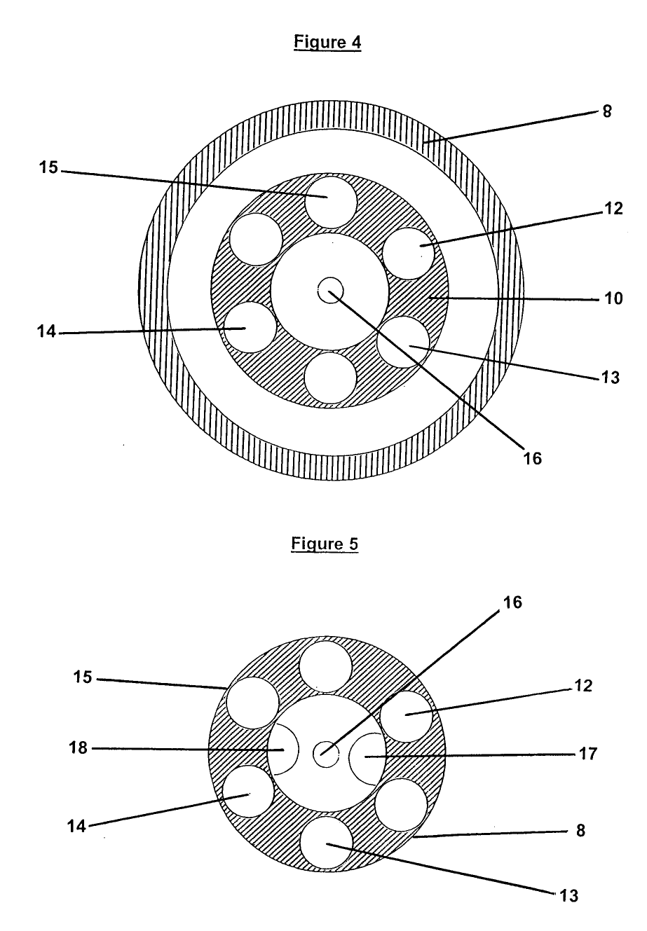

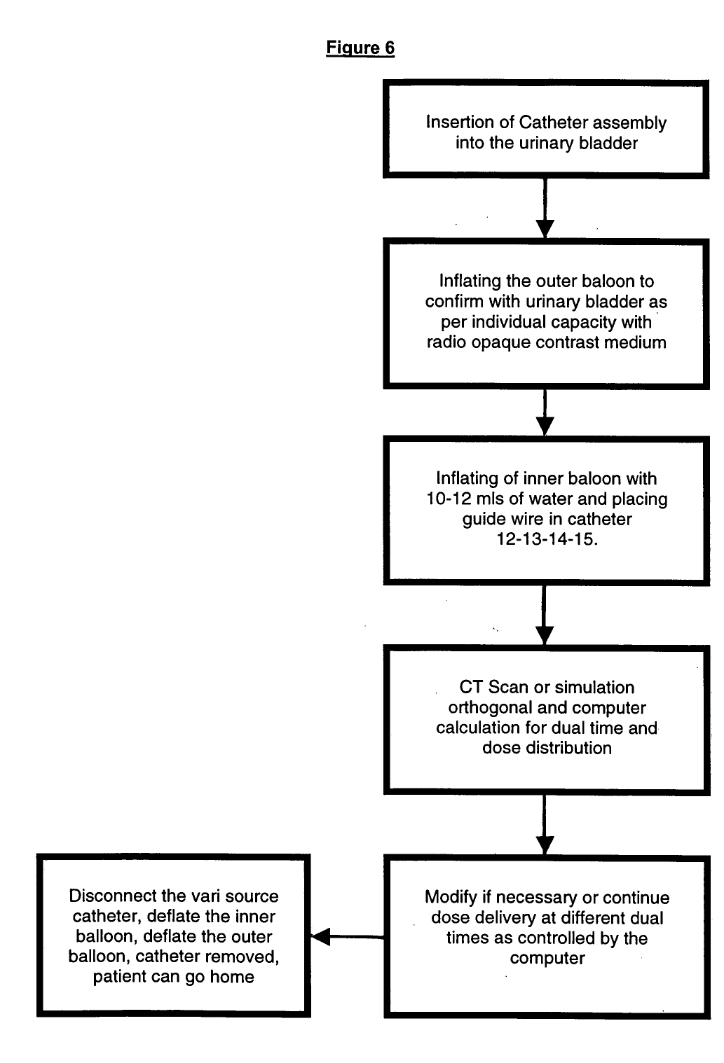

### EXAMPLE

In use the catheter, with balloons uninflated, is passed into the urinary bladder after suitable lubrication.

The outer balloon is then inflated. This has the effect of enlarging the bladder such that it snugly fits around the surface of the outer balloon. This facilitates the subsequent targeted radiation dosing.

The inner balloon is then partly inflated in or around the centre of the outer balloon.

The brachytherapy tubes are fused to the surface of the inner balloon and their cavity ends terminate at this upper end.

The free outer ends of each brachytherapy tube terminate with a Luer lock, which can be attached to a radioactive source loader of known type.

For imaging purposes a “dummy” wire is first inserted into the tubes to check the smooth passage therein. A CT (computerized tomography) scan is taken to check the positions of the dummy sources and permit the computer calculation of the dose distribution at the surface of the large balloon (i.e. the inner surface of the bladder) and the thickness of the bladder wall. Once this is completed the sources are attached to the Varisource machine (or to any other commercial radiation delivery machine) and necessary exposure is given to achieve the predetermined doses calculated by the computer and as approved by the Physician Oncologist. After completion of the dose delivery, the whole catheter is deflated by removing the fluid used to inflate it, and the catheter is removed.

1. An apparatus for applying therapeutic radiation emitted from a radiation source to a predetermined region of tissue within a viscous organ cavity in the body of a living being, said apparatus comprising:
(a) a flexible catheter for providing an access passageway into the body, which catheter has a distal end and a proximal end,
(b) one or more brachytherapy tubes carried within the catheter for receiving the radioactive source at the proximal end of the catheter and transferring the source into the cavity,
(c) an outer inflatable balloon element mounted at the distal end of the catheter for securing the catheter in the cavity and engaging the internal surface of the cavity and optionally distending the inner surface of the cavity,
(d) an inner inflatable balloon element mounted within the outer balloon element for positioning the brachytherapy tubes within the outer balloon element,
(e) a first fluid tube carried within the catheter for inflating the outer balloon element,
(f) a second fluid tube carried within the catheter for inflating the inner balloon element.

2. An apparatus as claimed in claim 1 wherein the first and second fluid tubes are also suitable for deflation of their respective balloons.

3. An apparatus as claimed in claim 1 wherein the organ is selected from the bladder, lungs, rectum or stomach.

4. An apparatus as claimed in claim 3 wherein the organ is the bladder.

5. An apparatus as claimed in claim 1 wherein the flexible catheter has an external diameter of about 8 mm.

6. An apparatus as claimed in claim 1 wherein the or each brachytherapy tube consists of a fine catheter having an internal diameter of between 0.6-1.0 mm.

7. An apparatus as claimed in claim 1 wherein the or each brachytherapy tube is present in the flexible catheter wall.

8. An apparatus as claimed in claim 1 wherein the or each brachytherapy tube terminates at its proximal end in attachment-means suitable for engaging a radioactive source loader.

9. An apparatus as claimed in claim 1 wherein there are 3, 4, 5, 8, 10 or more brachytherapy tubes.

10. An apparatus as claimed in claim 1 wherein the outer balloon is inflatable to a volume sufficient to contact and enlarge or distend the organ.

11. An apparatus as claimed in claim 1 wherein the outer balloon is inflatable to 300, 400, or 500 ml or more.

12. An apparatus as claimed in claim 1 wherein the outer balloon comprises radio-opaque material.

13. An apparatus as claimed in claim 1 wherein the inner balloon is inflatable to between 2-10 ml.

14. An apparatus as claimed in claim 1 wherein the first and second fluid tubes terminate at their proximal end in attachment-means suitable for engaging a fluid source under pressure.

15. A kit for applying therapeutic radiation emitted from a radiation source to a predetermined region of tissue within a viscous organ cavity in the body of a living being, said kit comprising:
flexible catheter for providing an access passageway into the body, which catheter has an distal end and a proximal end,
(b) one or more brachytherapy tubes carried within the catheter for receiving the radioactive source at the proximal end of the catheter and transferring the source into the cavity,
(c) a first fluid tube carried within the catheter for inflating an outer balloon element,
(d) a second fluid tube carried within the catheter for inflating an inner balloon element, plus one or both of:
(e) an outer inflatable balloon element for mounting at the distal end of the catheter for securing the catheter in the cavity and engaging and optionally distending the internal surface of the cavity,
(f) an inner inflatable balloon element mounted within the outer balloon element for positioning the brachytherapy tubes within the outer balloon element.

16. A kit as claimed in claim 15 wherein the first and second fluid tubes are also suitable for deflation of their respective balloons.

17. A kit as claimed in claim 15 or claim 16 wherein the organ is selected from the bladder, lungs, rectum or stomach.

18. A kit as claimed in claim 17 wherein the organ is the bladder.

19. A kit as claimed in claim 15 wherein the flexible catheter has an external diameter of about 8 mm.

20. A kit as claimed in claim 15 wherein the or each brachytherapy tube consists of a fine catheter having an internal diameter of between 0.6-1.0 mm.

21. A kit as claimed in claim 15 wherein the or each brachytherapy tube is present in the flexible catheter wall.

22. A kit as claimed in claim 15 wherein the or each brachytherapy tube terminates at its proximal end in attachment-means suitable for engaging a radioactive source loader.

23. A kit as claimed in claim 15 wherein there are 3, 4, 5, 8, 10 or more brachytherapy tubes.

24. A kit as claimed in claim 15 wherein the outer balloon is inflatable to a volume sufficient to contact and enlarge or distend the organ.

25. A kit as claimed in claim 15 wherein the outer balloon is inflatable to 300, 400, or 500 ml or more.

26. A kit as claimed in claim 15 wherein the outer balloon comprises radio-opaque material.

27. A kit as claimed in claim 15 wherein the inner balloon is inflatable to between 2-10 ml.

28. A kit as claimed in claim 15 wherein the first and second fluid tubes terminate at their proximal end in attachment-means suitable for engaging a fluid source under pressure.

29. A method of applying therapeutic radiation within a viscous organ cavity within the body of a living being, said method comprising:
(i) providing an apparatus as claimed in claim 1,
(ii) inserting the distal end of the catheter into a viscous organ cavity,
(iii) inflating the outer balloon such that its outer surface contacts the internal surface of the organ cavity,
(iv) partially inflating the inner balloon such as to distribute the or each brachytherapy tube within the outer balloon,
(v) transferring a radioactive source to a pre-determined position within the or each brachytherapy tube in the organ cavity for a pre-determined period of time such as to provide a targeted dosage of radiation to all or part of the internal surface of the organ cavity and/or the wall thickness of this organ cavity.

30. An apparatus as claimed in claim 1 or a kit including the same, for use in a method of treatment of the human body.

31. An apparatus or kit as claimed in claim 30 wherein the treatment is for bladder cancer.
